(19) United States
(12) Patent Application Publication
(10) Pub. No.: US 2023/0092415 A1
(43) Pub. Date: Mar. 23, 2023

(54) OIL-IN-OIL EMULSIFIED COSMETIC

(71) Applicant: Shiseido Company, Ltd., Chuo-ku, Tokyo (JP)

(72) Inventors: Misa KANAZAWA, Tokyo (JP); Yurie NISHIUMI, Tokyo (JP); Kiriko CHIBA, Tokyo (JP)

(73) Assignee: Shiseido Company, Ltd., Chuo-ku, Tokyo (JP)

(21) Appl. No.: 17/798,217

(22) PCT Filed: Feb. 19, 2021
(86) PCT No.: PCT/JP2021/006242

§ 371 Date: Aug. 8, 2022

(30) Foreign Application Priority Data
Feb. 20, 2020 (JP) 2020-026727

(51) Int. Cl. A61K 8/04; A61Q 1/04; A61K 8/02; A61K 8/92

(52) U.S. Cl. CPC A61K 8/04; A61Q 1/04; A61K 8/025; A61K 8/92; A61K 2800/412; A61K 2800/594; A61K 2800/92

(57) ABSTRACT

The purpose of the present invention is to provide an oil-in-oil emulsified cosmetic having exceptional transfer resistance after application, luster, excellent spread during application, and especially exceptional cosmetic durability. The present invention pertains to an oil-in-oil emulsified cosmetic characterized by containing (a) 5-80 mass % of a nonvolatile hydrocarbon oil and/or nonvolatile ester oil, (b) 1-70 mass % of a nonvolatile silicone oil, and (c) 2-12 mass % of a spherical powder having an average particle size of 1-10 μm, the oil-in-oil emulsified cosmetic moreover being characterized in that the (a) nonvolatile hydrocarbon oil and/or nonvolatile ester oil and the (b) nonvolatile silicone oil separate when mixed at 25° C. The blend ratio (mass ratio) of the (a) nonvolatile hydrocarbon oil and/or nonvolatile ester oil and the (b) nonvolatile silicone oil is preferably (a)/[(a)+(b)]=0.4-0.8.

### TECHNICAL FIELD

The present invention relates to an oil-in-oil type emulsified cosmetic and more particularly to an oil-in-oil type emulsified cosmetic that is excellent in transfer resistance, spreads well at the time of application, and is also excellent in makeup durability.

### BACKGROUND ART

Lip cosmetics such as lipsticks have the problem of secondary adhesion in which after being applied to the lips, the cosmetics are transferred to substances that come into contact with the lips, such as food and cups. There are also cases where a beautiful color immediately after application is lost due to secondary adhesion or a color changes over time, and there is a need for lip cosmetics excellent in makeup durability in which a makeup effect lasts for a long time, in addition to no secondary adhesion.

In order to suppress secondary adhesion (improve transfer resistance) or improve makeup durability in lip cosmetics, various techniques are developed. Typical examples include a technique in which a volatile oil such as a cyclic silicone oil, and a film-forming agent (silicone-based resin) dissolved in the volatile oil are blended into a lip cosmetic, and after application to the lips, secondary adhesion is prevented by a film formed by the evaporation of the volatile oil. However, a problem has been that an uncomfortable feeling (tight feeling) occurs due to the film formed on the lips. There is also a technique in which a lipstick is applied, and a lip coat is reapplied thereon to prevent color transfer and color loss, but the technique has needed double application and has been complicated.

There are also proposed ideas for suppressing secondary adhesion in lip cosmetics such as lipsticks to improve makeup durability, and, for example, Patent Document 1 discloses a lip cosmetic that is glossy and excellent in color durability (makeup durability) and a feeling of application (smoothness) by blending a particular amino-modified silicone, an oil having a particular IOB value, and polybutene in combination.

Patent document 2 discloses a nonsolid lip cosmetic that is excellent in a transparent feeling and gloss at the time of application, has no stickiness, and is also excellent in makeup durability by blending a high viscosity oil and a semisolid oil at a predetermined ratio and including pentaerythrityl tetra(behenate/benzoate/2-ethylhexanoate) in the semisolid oil.

Patent document 3 describes an oil-in-oil type cosmetic including (a) a nonvolatile hydrocarbon oil as a continuous phase and (b) a nonvolatile silicone oil as a dispersed phase, wherein the amounts of the (a) nonvolatile hydrocarbon oil and the (b) nonvolatile silicone oil and their amount ratio are adjusted within particular ranges, and furthermore a dextrin fatty acid ester is blended. It is stated that when the cosmetic of Patent Document 3 is applied to the lips, the nonvolatile silicone oil seeps to the surface layer (bleeding phenomenon) and forms a coat layer coating the adhesive layer of the nonvolatile hydrocarbon oil, and therefore the cosmetic of Patent Document 3 is excellent in transfer resistance and furthermore gives a good gloss. However, in terms of makeup durability, there has been room for improvement in the conventional art.
Patent Document 1: JP-A 2007-176866
Patent Document 2: JP-B 4786399
Patent Document 3: JP-B 4766720

### DISCLOSURE OF INVENTION

### Technical Problem

Accordingly, it is an object of the present invention to provide an oil-in-oil type cosmetic that is excellent in transfer resistance after application, spreads well at the time of application, provides a glossy finish, and furthermore is also excellent in makeup durability.

### Solution to Problem

As a result of diligent studies, the present inventors have found that a cosmetic that spreads well at the time of application, is excellent in transfer resistance after application, and furthermore is excellent in makeup durability is obtained by blending a predetermined amount of a spherical powder having an average particle diameter in a predetermined range into an oil-in-oil type emulsified cosmetic containing a nonvolatile hydrocarbon oil (and/or nonvolatile ester oil) and a nonvolatile silicone oil that are not compatible with each other. Thus, the present inventors have completed the present invention.

Specifically, the present invention provides

an oil-in-oil emulsified cosmetic comprising:

(a) 5 to 80% by mass of a nonvolatile hydrocarbon oil and/or nonvolatile ester oil;

(b) 1 to 70% by mass of a nonvolatile silicone oil; and

(c) 2 to 12% by mass of a spherical powder having an average particle diameter of 1 to 10 μm,

wherein the (a) nonvolatile hydrocarbon oil and/or nonvolatile ester oil and the (b) nonvolatile silicone oil separate when mixed at 25° C.

In the present invention, a blending amount ratio (mass ratio) between the (a) nonvolatile hydrocarbon oil and/or nonvolatile ester oil and the (b) nonvolatile silicone oil, which is represented by “(a)/[(a)+(b)]”, is preferably in a range from 0.4 to 0.8.

### Advantageous Effects of Invention

The oil-in-oil emulsified cosmetic of the present invention is excellent in transfer resistance after application, spreads well at the time of application, provides a glossy finish even when powders are blended, and furthermore is also excellent in makeup durability.

### DESCRIPTION OF EMBODIMENTS

The oil-in-oil emulsified cosmetic (also referred to as an “oil-in-oil cosmetic” or simply as an “emulsified cosmetic”) of the present invention contains (a) a nonvolatile hydrocarbon oil and/or nonvolatile ester oil, (b) a nonvolatile silicone oil, and (c) a spherical powder having a particular particle diameter, as essential components. A description will be given in detail below.

(a) Nonvolatile Hydrocarbon Oil and/or Nonvolatile Ester Oil

The nonvolatile hydrocarbon oil and/or nonvolatile ester oil used in the present invention include at least one oil selected from (a1) a nonvolatile hydrocarbon oil and (a2) a nonvolatile ester oil. The “nonvolatile oil” herein is a liquid or pasty oil that does not exhibit volatility at normal temperature (25° C.) and normal pressure.

(a1) Nonvolatile Hydrocarbon Oil

Examples of the (a1) nonvolatile hydrocarbon oil include hydrogenated polyisobutene, polybutene, polyisobutylene, polyisoprene, liquid paraffins, squalane, hydrogenated polydecene, and petrolatum (Vaseline). Among these, polybutene is preferred, and in particular, polybutene having a molecular weight of 1000 to 2650 is further preferred.

(a2) Nonvolatile Ester Oil

Examples of the (a2) nonvolatile ester oil include dipentaerythrityl hexahydroxystearate (trade name: COSMOL 168M), dimer dilinoleates such as (polyglyceryl-2 isostearate/dimer dilinoleate) copolymer (trade name: HAILUCENT ISDA), dimer dilinoleyl dimer dilinoleate (trade name: LUSPLAN DD-DA7), and (phytosteryl/isostearyl/cetyl/stearyl/behenyl) dimer dilinoleate, and lauroyl glutamates such as di(octyldodecyl/phytosteryl/behenyl) lauroyl glutamate and dimer dilinoleyl bis(lauroyl glutamate/lauroyl sarcosinate). Among these, particularly, dipentaerythrityl hexahydroxystearate, (polyglyceryl-2 isostearate/dimer dilinoleate) copolymer, and the like are preferably used.

The amount of the (a) nonvolatile hydrocarbon oil and/or nonvolatile ester oil in the emulsified cosmetic of the present invention is 5 to 80% by mass, preferably 10 to 70% by mass, and more preferably 20 to 60% by mass based on the total amount of the cosmetic. When the amount of the nonvolatile hydrocarbon oil is too small, moistness is lacking. When the amount of the nonvolatile hydrocarbon oil is too large, the spread tends to be heavy, the stickiness tends to increase, the transfer resistance tends to deteriorate, and the coloring (or chromogenic) effect also tends to poor.

(b) Nonvolatile Silicone Oil

The (b) nonvolatile silicone oil should be not compatible with the nonvolatile hydrocarbon oil and the nonvolatile ester oil and is appropriately selected so as to form an oil-in-oil emulsion according to the type of the hydrocarbon oil simultaneously blended. Examples of such a nonvolatile silicone oil include diphenyl dimethicone (also referred to as methylphenylpolysiloxane), dimethicone, and fluorine-modified alkyl silicones. Among these, diphenyl dimethicone is particularly preferred, and diphenyl dimethicone having a viscosity of 300 to 500 cs (cSt) is further preferred.

The amount of the (b) nonvolatile silicone oil in the emulsified cosmetic of the present invention is 1 to 70% by mass, preferably 5 to 60% by mass, and more preferably 10 to 50% by mass based on the total amount of the cosmetic. When the amount of the nonvolatile silicone oil is too small, the transfer resistance tends to deteriorate. When the amount of the nonvolatile silicone oil is too large, the gloss increases, but the emulsified cosmetic comes off easily over time.

“Separate(s)” or “not compatible” (at 25° C.) herein is defined as follows: The (a) nonvolatile hydrocarbon oil and/or nonvolatile ester oil and the (b) nonvolatile silicone oil are used at (a):(b)=1:1 (mass ratio), heated to 90° C., stirred and mixed, and then allowed to stand. A mixture that separates into two layers, the boundary between which is uniform, when the temperature of the mixture reaches 25° C. is considered “separate(s)” or “not compatible”, and a mixture that reaches a semitransparent state, or a transparent compatible state without a boundary, when the temperature of the mixture reaches 25° C. is considered “do(es) not separate” or “compatible”.

In the emulsified cosmetic of the present invention, the blending ratio (mass ratio) between the (a) nonvolatile hydrocarbon oil and/or nonvolatile ester oil and the (b) nonvolatile silicone oil is preferably (a)/[(a)+(b)]=0.4 to 0.8. When the amount of the (a) component is too large based on the [(a)+(b)] components, the spread is heavy, the stickiness increases, the transfer resistance deteriorates, and the coloring (chromogenic) effect also becomes poor. When the amount of the (a) component is too small, the proportion of (b) in (a)+(b) increases, and thus moistness tends to be lacking.

(c) Spherical Powder Having Particular Particle Diameter

The (c) component in the emulsified cosmetic of the present invention is a spherical powder having an average particle diameter of 1 to 10 μm (referred to as a “spherical powder having a particular particle diameter”). The average particle diameter is more preferably 2 to 8 μm, further preferably 3 to 7 μm. With a powder having an average particle diameter of less than 1 μm, or a powder having an average particle diameter of more than 10 μm, the effect of improving makeup durability is not obtained. The “average particle diameter” herein means the arithmetic mean value of particle diameters measured for a plurality of the particles of the powder (that is, the value of D50 in the particle size distribution).

The shape of the powder having a particular particle diameter in the present invention needs to be spherical. The spherical powder is a powder in which the length of the major axis to the length of the minor axis is 2 or less, preferably 1.5 or less, and is most preferably a powder having a true spherical shape. When the powder shape is not spherical (is plate-type, flake-type, or the like), there are cases where the powder does not fit with the nonvolatile silicone oil and settles.

The material of the spherical powder is not particularly limited, and inorganic powders such as silica, and inorganic or organic resin powders such as polymethylsilsesquioxane can be used. The powder surface may be hydrophobic or hydrophilic, but a spherical powder having a hydrophobic surface is preferably used.

In the emulsified cosmetic of the present invention, the spherical powder having a particular particle diameter is preferably blended into the dispersed phase (internal phase).

The amount of the (c) spherical powder having a particular particle diameter in the emulsified cosmetic of the present invention is 2 to 12% by mass, preferably 3 to 10% by mass, based on the total amount of the cosmetic. When the amount is less than 2% by mass, the effect of improving makeup durability cannot be exhibited. When more than 12% by mass of the (c) spherical powder having a particular particle diameter is blended, there are cases where it does not fit with the nonvolatile silicone oil and settles.

Like the cosmetic described in Patent Document 3, the emulsified cosmetic of the present invention is an oil-in-oil type emulsified cosmetic including a nonvolatile hydrocarbon oil and/or nonvolatile ester oil as a continuous phase and a nonvolatile silicone oil as a dispersed phase that are not compatible with each other. It is considered that in the oil-in-oil type emulsified cosmetic of the present invention, a color material such as iron oxide disperses in the continuous phase oil due to the wettability (affinity) of the surface. In Patent Document 3, it is shown that emulsification stability cannot be maintained unless a dextrin fatty acid ester, which is an oily thickening agent, is blended. But in the present invention, even if a dextrin fatty acid ester is blended, by blending the spherical powder having a particular particle diameter, the viscosity increases, and the emulsification stability can be maintained, and the makeup durability can be improved, which are advantageous effects unpredictable from conventional art.

In the oil-in-oil type cosmetic of the present invention, the (b) nonvolatile silicone oil and the (a) nonvolatile hydrocarbon oil and/or ester oil maintain an oil-in-oil type stable emulsified state in a container before use. On the other hand, when the oil-in-oil type cosmetic of the present invention is applied to the lips or the like, demulsification occurs due to the external force applied at the time of application, and the (b) nonvolatile silicone oil seeps to the surface layer, separates, and forms a coat layer. It covers the adhesive layer of the (a) nonvolatile hydrocarbon oil and/or nonvolatile ester oil, and therefore the oil-in-oil type cosmetic of the present invention has transfer resistance and gives a good gloss. This separation of the (b) nonvolatile silicone oil is further promoted by rubbing the lips together and applying pressure at the time of application.

That is, when the emulsified cosmetic of the present invention is used, for example, as a lipstick, the following effect is exhibited: there is no trouble of using two articles such as a lipstick and a lip coat, and with one article, the color is less likely to be lost and less likely to be attached to a cup or the like.

In addition to the essential components (a), (b), and (c) above, other components usually blended into makeup cosmetics, especially lip cosmetics such as lipsticks, can be blended into the emulsified cosmetic of the present invention in a range that does not impair the effect of the present invention. Other components that can be used will be illustrated below.

(d) Volatile Hydrocarbon Oil

When a volatile hydrocarbon oil such as isododecane is blended into the emulsified cosmetic of the present invention, the spread at the time of application is further improved. As the volatile hydrocarbon oil, those soluble in (or compatible with) both the (a) nonvolatile hydrocarbon oil and/or nonvolatile ester oil and the (b) nonvolatile silicone oil are preferred. For example, volatile hydrocarbon oils having 8 to 16 carbon atoms and mixtures thereof can be illustrated. The volatile hydrocarbon oil is preferably selected particularly from branched C8-C16 alkanes, branched C8-C16 esters, and mixtures thereof. Preferred as such a volatile hydrocarbon oil are particularly C8-C16 isoparaffins obtained from petroleum, and commercial products include “Isopar” (isoparaffin-based solvent, manufactured by Exxon Mobil) and “Permethyl 99A, Permethyl 101A” (manufactured by EC Eldorchemie, sold by NIHON KOKEN KOGYO CO., LTD.). For illustrations of compound names, isododecane, isohexadecane, isohexyl neopentanoate, and mixtures thereof are preferred, and isododecane is particularly preferred.

The preferred amount of the (d) volatile hydrocarbon, when it is blended into the emulsified cosmetic of the present invention, is 0.1 to 50% by mass, preferably 1 to 30% by mass, and more preferably 2 to 20% by mass based on the total amount of the cosmetic.

(e) Coloring Material

The emulsified cosmetic of the present invention is suitable for use as makeup cosmetics, especially lip cosmetics such as lipsticks. Therefore, a coloring material is preferably blended into the emulsified cosmetic of the present invention.

The coloring material blended in the present invention should be a coloring material usually used in makeup cosmetics such as lipsticks, and includes pigments (powders or lakes) and dyes. Inorganic pigments, organic pigments, or pearl pigments all tend to be wetted with hydrocarbon oils easier than with silicone oils, and therefore finally the pigment spontaneously migrates to the hydrocarbon oil, which is the continuous phase.

The amount of the coloring material in the emulsified cosmetic of the present invention is usually 0.01 to 30% by mass, preferably 0.1 to 20% by mass, based on the total amount of the cosmetic.

(f) Powder Other than (c) Component

The emulsified cosmetic of the present invention may contain, in addition to the (c) spherical powder having a particular particle diameter, a powder not applicable to the (c) component.

For example, silicic anhydride (silica) having an average particle diameter of 10 to 50 nm is preferably blended. As used herein, these fine particles having an average particle diameter on the order of nanometers are referred to as “nanoparticles”. As nanoparticles of silicic anhydride (silica), commercial products such as AEROSIL 200, 300, R972, R974, and RY200 (manufactured by NIPPON AEROSIL CO., LTD.) can be used. The silicic anhydride (silica) used in the present invention may be silicic anhydride (silica) having a hydrophilic surface or silicic anhydride (silica) subjected to hydrophobization treatment such as silylation. The preferred amount of the silicic anhydride (silica) nanoparticles, when they are blended, is preferably 10% by mass or less, more preferably 0.1 to 5% by mass based on the total amount of the cosmetic.

(g) Oily Thickening Agent

An oily thickening agent that is oil-soluble and has a thickening action can be blended into the emulsified cosmetic of the present invention. Especially, dextrin fatty acid esters are mentioned as preferred examples. The amount of the oily thickening agent is preferably 0.1 to 8% by mass, more preferably 0.5 to 6% by mass, and further preferably 1 to 5% by mass based on the total amount of the cosmetic. In the emulsified cosmetic of the present invention, the emulsification stability is maintained even if a dextrin fatty acid ester is not blended, and therefore the present invention also includes a form in which a dextrin fatty acid ester is not blended.

(h) Other Components

Examples of other components blended into the emulsified cosmetic of the present invention include, but are not limited to, a moisturizer, a semisolid oil (other than the (a) component) and/or a microcrystalline wax, a perfume, a surfactant, an antioxidant, a preservative, and a beauty component.

As the moisturizer, polyhydric alcohol-based moisturizers such as glycerin, propylene glycol, and 1,3-butylene glycol are illustrated.

When too large amounts of a wax (other than a microcrystalline wax) and a film-forming agent are blended, a decrease in the gloss of a coating, and a stiff feeling may be caused. Therefore, the amounts of these are preferably suppressed to about 5% by mass or less, and the present invention also includes a mode in which a wax (other than a microcrystalline wax) and a film-forming agent are not blended.

The emulsified cosmetic of the present invention can be produced, for example, by stirring and mixing the constituents of the continuous phase including the (a) nonvolatile hydrocarbon oil and/or nonvolatile ester oil while heating them as needed, separately stirring and mixing the constituents of the dispersed phase including the (b) nonvolatile silicone oil and the (c) spherical powder having a particular particle diameter while optionally heating them, and stirring and dispersing the dispersed phase into the continuous phase with stirring.

The emulsified cosmetic of the present invention can be applied to lipsticks, lip glosses, lip bases for base coat, lipstick overcoats, lip creams, and the like. Particularly, when the emulsified cosmetic of the present invention is used for a lipstick in which a color material is blended, it can have a good coloring (chromogenic) effect and good transfer resistance as a lipstick and be excellent particularly in makeup durability and is therefore preferred.

### EXAMPLES

The present invention will be described in more detail below by giving Examples, but the present invention is not limited in any way by these examples. The amounts are expressed in % by mass unless otherwise specified.

Prior to the description of the Examples, the evaluation test method used in the present invention will be described.

Evaluation Test Method

An actual usability test by 10 expert panelists was performed. The usability items were spread at the time of application, gloss, transfer resistance, and makeup durability, and for each evaluation item, five-grade sensory evaluation (scoring) was performed based on the following evaluation point criteria. From the score average value, a determination was made according to the following evaluation criteria.

The evaluation of spread at the time of application, gloss, and transfer resistance is evaluation at the time of application or immediately after application, whereas for makeup durability, the persistence of a makeup effect at 2 hours after application is evaluated. For the application method, application was performed by a method of applying the cosmetic of the present invention to the lips, then rubbing the upper and lower lips together, and applying pressure for about 5 seconds. For the evaluation of transfer resistance, the lack of transfer to a cup was evaluated, and for the evaluation of makeup durability, the makeup durability was determined by whether a lipstick-applied state was maintained after 2 hours.

(Scores)

5 points: Superior.

4 points: Excellent.

3 points: Average.

2 points: Poor.

1 point: Very poor.

(Evaluation Criteria)

S: an evaluation value (average value) of 4.0 or more

A: an evaluation value (average value) of 3.0 or more and less than 4.0 points

B: an evaluation value (average value) of 2.0 or more and less than 3.0 points

C: an evaluation value (average value) of less than 2.0 points

### Examples 1 to 6 and Comparative Examples 1 to 7

Gloss lipsticks were prepared with the formulations shown in the following Tables 1 and 2 and evaluated for usability (spread at the time of application, gloss, transfer resistance) and makeup durability according to the criteria. The results are shown together in Tables 1 and 2.

The (numerical values) given to the powders in the tables are average particle diameters. The portions of “-” for the evaluation results mean that the powder did not fit (settling or the like occurred), and the measurement could not be performed.

TABLE 1

| | Comparative | Comparative | | | | Comparative |
|---|---|---|---|---|---|---|
| | Example | Example | Example | Example | Example | Example |
| | 1 | 2 | 1 | 2 | 3 | 3 |
| Hydrogenated polyisobutene | Balance | Balance | Balance | Balance | Balance | Balance |
| (average molecular weight 1000) | | | | | | |
| Hydrogenated polyisobutene | 5 | 5 | 5 | 5 | 5 | 5 |
| (average molecular weight 2650) | | | | | | |
| Color material | 2 | 2 | 2 | 2 | 2 | 2 |
| Dimethylsilylated silica | 1 | 1 | 1 | 1 | 1 | 1 |
| (nanoparticles) | | | | | | |
| Vaseline | 10 | 10 | 10 | 10 | 10 | 10 |
| (Phytosteryl/behenyl) dimer | 5 | 5 | 5 | 5 | 5 | 5 |
| dilinoleate | | | | | | |
| Microcrystalline wax | 2 | 2 | 2 | 2 | 2 | 2 |
| Isododecane | 6.7 | 6.7 | 6.7 | 6.7 | 6.7 | 6.7 |
| Dextrin palmitate | 3.3 | 3.3 | 3.3 | 3.3 | 3.3 | 3.3 |
| Dipropylene glycol | 0.2 | 0.2 | 0.2 | 0.2 | 0.2 | 0.2 |
| Diphenyl dimethicone | 35 | 35 | 35 | 35 | 35 | 35 |
| Silica (nanoparticles) | 1 | 1 | 1 | 1 | 1 | 1 |
| (Vinyl dimethicone/methicone | — | 1 | 2 | 5 | 10 | 15 |
| silsesquioxane) crosspolymer | | | | | | |
| (3~8) (*) | | | | | | |
| Total (% by mass) | 100 | 100 | 100 | 100 | 100 | 100 |
| (a)/[(a) + (b)] | 0.49 | 0.49 | 0.48 | 0.45 | 0.41 | 0.35 |
| Viscosity (mPa · s) | 14000 | 28300 | 25400 | 11200 | 15900 | — |
| Spread | S | S | S | S | S | — |
| Transfer resistance | S | S | S | S | S | — |
| Gloss | S | S | S | S | S | — |
| Makeup durability | B | B | A | A | A | — |

(*): a spherical powder having a particular particle diameter

TABLE 2

| | | | Comparative | Comparative | Comparative | Comparative | |
|---|---|---|---|---|---|---|---|
| | Example | Example | Example | Example | Example | Example | Example |
| | 4 | 5 | 4 | 5 | 6 | 7 | 6 |
| Hydrogenated polyisobutene | Balance | Balance | Balance | Balance | Balance | Balance | — |
| (average molecular weight 1000) | | | | | | | |
| Hydrogenated polyisobutene | 5 | 5 | 5 | 5 | 5 | 5 | — |
| (average molecular weight 2650) | | | | | | | |
| (Polyglyceryl-2 isostearate/dimer | — | — | — | — | — | — | Balance |
| dilinoleic acid) copolymer | | | | | | | |
| Color material | 2 | 2 | 2 | 2 | 2 | 2 | 2 |
| Dimethylsilylated silica | 1 | 1 | 1 | 1 | 1 | 1 | 1 |
| (nanoparticles) | | | | | | | |
| Vaseline | 10 | 10 | 10 | 10 | 10 | 10 | 10 |
| (Phytosteryl/behenyl) dimer | 5 | 5 | 5 | 5 | 5 | 5 | 5 |
| dilinoleate | | | | | | | |
| Microcrystalline wax | 2 | 2 | 2 | 2 | 2 | 2 | 2 |
| Isododecane | 6.7 | 6.7 | 6.7 | 6.7 | 6.7 | 6.7 | 6.7 |
| Dextrin palmitate | — | 3.3 | 3.3 | 3.3 | 3.3 | 3.3 | 3.3 |
| Dipropylene glycol | 0.2 | 0.2 | 0.2 | 0.2 | 0.2 | 0.2 | 0.2 |
| Diphenyl dimethicone | 35 | 35 | 35 | 35 | 35 | 35 | 35 |
| Silica (nanoparticles) | 1 | 1 | 1 | 1 | 1 | 1 | 1 |
| (Vinyl dimethicone/methicone | 10 | — | — | — | — | — | 10 |
| silsesquioxane) crosspolymer | | | | | | | |
| (3~8) (*) | | | | | | | |
| Silica (1.5~2.5) (*) | — | 2.5 | — | — | — | — | — |
| Silica (nanoparticles) (***) | — | — | 3 | — | — | — | — |
| Synthetic phlogopite (9~14) (**) | — | — | — | 10 | — | — | — |
| Mica (5) (**) | — | — | — | — | 10 | — | — |
| (HDI/trimethylol hexyllactone) | — | — | — | — | — | 10 | — |
| crosspolymer (12~18) (***) | | | | | | | |
| Total (% by mass) | 100 | 100 | 100 | 100 | 100 | 100 | 100 |
| (a)/[(a) + (b)] | 0.43 | 0.47 | 0.47 | 0.41 | 0.41 | 0.41 | 0.43 |
| Viscosity (mPa · s) | 46800 | 20700 | — | — | — | — | 39800 |
| Spread | S | S | — | — | — | — | S |
| Transfer resistance | S | S | — | — | — | — | S |
| Gloss | S | S | — | — | — | — | S |
| Makeup durability | A | A | — | — | — | — | A |

(*): a spherical powder having a particular particle diameter
(**): a powder other than a spherical powder
(***): a spherical powder having an average particle diameter outside the predetermined range

From the results shown in Tables 1 and 2, Examples 1 to 6 which contain predetermined amount of the (c) spherical powder having a particular particle diameter were excellent in spread at the time of application, gloss, and transfer resistance and also had extremely good makeup durability. These results were the same when either a nonvolatile hydrocarbon oil or a nonvolatile ester oil was used as the (a) component. In contrast, when a spherical powder was not blended or blended in too small amount, the makeup durability was not sufficient; whereas, when the spherical powder was blended in too large amount, the powder did not fit with the silicone oil and settling occurred. When a powder that was not spherical was used or when a powder having an average particle diameter outside the predetermined range was used, the powder did not fit with the silicone oil, and the intended emulsified cosmetic was not obtained.

1. An oil-in-oil emulsified cosmetic comprising:
(a) 5 to 80% by mass of a nonvolatile hydrocarbon oil and/or nonvolatile ester oil;
(b) 1 to 70% by mass of a nonvolatile silicone oil; and
(c) 2 to 12% by mass of a spherical powder having an average particle diameter of 1 to 10 μm, wherein said (a) nonvolatile hydrocarbon oil and/or nonvolatile ester oil and said (b) nonvolatile silicone oil separate when mixed at 25° C.

2. The cosmetic according to claim 1, wherein an amount ratio (mass ratio) between said (a) nonvolatile hydrocarbon oil and/or nonvolatile ester oil and said (b) nonvolatile silicone oil is (a)/[(a)+(b)]=0.4 to 0.8.

3. The cosmetic according to claim 1, further comprising (d) a volatile hydrocarbon.

4. The cosmetic according to claim 1, further comprising (e) a coloring material.

5. The cosmetic according to claim 1, further comprising (f) silicic anhydride nanoparticles.

6. A lip cosmetic comprising the cosmetic according to claim 1.

7. A lip cosmetic comprising the cosmetic according to claim 2.

8. A lip cosmetic comprising the cosmetic according to claim 3.

8. A lip cosmetic comprising the cosmetic according to claim 4.

10. A lip cosmetic comprising the cosmetic according to claim 5.
